(19) United States
(12) Patent Application Publication
(10) Pub. No.: US 2023/0173210 A1
(43) Pub. Date: Jun. 8, 2023

(54) MEDICAL VENTILATION MASK

(71) Applicant: INSPIR LABS LTD., Migdalim (IL)

(72) Inventors: Nadav Nahmias, Tel Aviv (IL); Eliyahu Shwalm, Migdalim (IL)

(21) Appl. No.: 17/924,519

(22) PCT Filed: Jun. 3, 2021
(86) PCT No.: PCT/IL2021/050657

§ 371 Date: Nov. 10, 2022

(30) Foreign Application Priority Data
Jun. 3, 2020 (IL) 275097
May 12, 2021 (IL) 283119

(51) Int. Cl. A61M 16/06; A61M 16/20

(52) U.S. Cl. CPC A61M 16/0616; A61M 16/208; A61M 16/0683

(57) ABSTRACT

An aerosol-proof ventilation mask which efficiently and safely collects and removes aerosol droplets. The medical ventilation mask comprising a gas opening, a mask frame having an internal portion and an external portion, an internal seal having an internal rim, an external seal having an external rim, and at least one vacuum outlet port connected to the mask frame to provide fluid communication with a vacuum line.

### TECHNOLOGICAL FIELD

The present invention is in the field of medical ventilating a subject, and more specifically to a ventilation mask for use in non-invasive ventilation (NIV).

### BACKGROUND

Medical ventilation is the procedure of mechanically supporting the breathing of a subject in need, by directly supplying oxygen through the airways of the subject (either mouth, throat, or nose) into the lungs and, at times, also removing carbon dioxide from the lungs. Ventilation is applied, inter alia on subjects who cannot breathe autonomously, or under surgery. Non-invasive ventilation (NIV) is commonly used to support subjects with chronic conditions, such as obstructive sleep apnea syndrome (OSAS), as well as acute conditions such as chronic obstructive pulmonary disease (COPD) exacerbation.

The Coronavirus (COVID-19) pandemic had led to a surged need for medical ventilations. As an attempt to resolve ventilators shortage, other respiratory devices were repurposed to ventilators. On March, 2020, The U.S. Food and Drug Administration (FDA) had issued a Letter to Health Care Providers in which it guides health care professionals to modify continuous positive airway pressure (CPAP) and bi-level positive air pressure (BiPAP) machines to treat respiratory insufficiency. Mainly, the guidance outlined a requirement to do so while preventing aerosolization of the virus. Aerosol is a suspension of liquid droplets in gas, such as air. Unlike larger droplets, aerosol may reside in the air for prolonged periods of time and lead to airborne infections. Thus, microbial aerosols, such as viral aerosols, are highly infectious. Aerosol is generated when high pressured air contacts saliva or nasal secretions carrying the pathogen. Common examples are coughing and sneezing, but NIV and inhalation also cause aerosol production. In some cases, aerosol drug is additionally administered during ventilation, thereby leading to the spreading of undesired, and at times, hazardous aerosol drug.

Given these complications, it was determined that the use of CPAP as medication requires manufacturer modifications and should be carefully monitored.

A CPAP machine is a mechanical ventilation device providing a continuous constant pressurizes air to a subject. It is currently known as a sleep apnea treatment, assisting subjects overnight by maintaining breathing airways unobstructed while sleeping. Similarly, a BiPAP machines delivers two air pressures, one for inhalation and a lower one to allow exhalation. When implementing a non-invasive device in subjects suffering from an infectious disease there is a considerable risk of environmental contamination. This risk is in fact aggravated when face-mask system, high-flow nasal cannula system, or a CPAP system is used. Specifically, the CPAP is an open system in which the exhaled air spreads in the environment. Accordingly, the FDA recommends acting precautionarily and applying negative pressure or additional filtration seal, where feasible, when using CPAP machines as ventilators, to avoid viral aerosolization, and subsequent disease spread. Studies carried out during the 2003 SARS epidemic, were concluded with a similar finding; CPAP devices pump viruses to the environment, contributing to disease spread by allowing air escape from the face mask.

In practice, professionals struggled to implement CPAP machines as ventilators without compromising on public health as CPAP are increasing the aerosol transmission in both hospitals and at home. The main identified difficulty was avoiding and removing viral aerosolization from the ventilation environment. The American Society of Anesthesiologists had even issued guidance discouraging CPAP use in COVID-19 subjects. Currently, a considerable number of subjects are consequently prevented from the benefits of such treatments, such as fewer intubation-related complications and high availability, to name a few.

Therefore, there is a growing need for non-invasive ventilation (NIV) solutions that are aerosol-proof and thus environmentally safe.

U.S. Pat. No. 4,895,172 discloses a collection device for collecting anesthetic gas exhaled by a subject. The device includes a chin-wearable member and an opening position near the subject's mouth.

International application publication no. WO 88/06044 discloses an anesthetic scavenging face mask comprising a respiratory chamber, a face seal, gas inlet, connection channel and vacuum outlet.

CN202724407U discloses pollution-preventing mask for inhaling anesthetic gas. The mask comprises a negative pressure suction connector connected with a negative pressure suction pipe for carrying out continuous negative pressure suction.

### GENERAL DESCRIPTION

Provided by this disclosure is an aerosol-proof ventilation mask which efficiently and safely collects and removes aerosol droplets.

The disclosed mask permits medical ventilation to a subject suffering from microbial disease, such as a viral disease, while keeping the environment of the ventilated subject safe by preventing aerosol in the exhaled breath from leaking outside of the ventilation system. The environmentally safe ventilation of this disclosure is enabled by collecting and removing aerosols exhaled by the subject in the course of ventilation. By scavenging viral aerosol to a vacuum port connectable to a vacuum source, environmentally safe ventilation is guaranteed.

This disclosure provides a solution for NIV ventilation, such as CPAP that involves aerosol collection and clearance. In CPAP ventilation the applied pressure remains relatively high and constant to permit medical treatment. These medical requirements make the clearance of the relatively heavier aerosol droplets during CPAP ventilation a highly challenging task. This disclosure guarantees a controlled vacuum-based removal of the aerosols during safe ventilation and obviates at least some of the limitations that direct suction that could potentially interfere with the ventilation itself and negate the benefits of NIV ventilation. The manner vacuum is utilized by some embodiments of this disclosure also ensures the sealing and improved scavenging of residual aerosols to prevent its spreading to the environment.

This disclosure provides, according to a first of its aspects, a medical ventilation mask having a central axis and comprising, at least when the mask is ready for use: a gas opening configured to be connected, in fluid communication to a ventilation machine and defining a location of a reference plane perpendicular to the central axis; a mask frame having an internal portion and an external portion and accommodating the gas opening so as to provide fluid communication of the gas opening with the internal portion, the external portion overlaying said internal portion at least along a part of an extension thereof along the central axis and including an auxiliary space therebetween; an internal seal connected to said internal portion at one end of the seal and having an internal rim at another end thereof, and configured for contacting with the face of a subject so that the internal portion of the mask frame and the internal seal define with the face of a subject a ventilation cavity in fluid communication with the gas opening for delivering pressurized ventilation gas to the airways of the subject, said internal rim being spaced from the reference plane along the central axis to a first distance D1; an external seal connected to said external portion at one end of the seal and having an external rim at another end thereof, and configured for contacting with the face of the subject at a location spaced from the internal rim in a direction perpendicular to the central axis, so that said external portion of the mask frame with its auxiliary space and the external seal define with the face of a subject a vacuum cavity, said external rim being spaced from the reference plane along the central axis to a second distance D2 longer than the first distance; and at least one vacuum outlet port connected to the mask frame so as to provide fluid communication between said auxiliary space and a vacuum line.

By some embodiments, the mask frame has an external wall and an internal wall spaced inwardly from the external wall in a direction perpendicular to the central axis, the external wall defining at least partially said external portion of the mask frame, and the internal wall defining at least partially said internal portion of the mask frame, and wherein the mask frame optionally further comprises a cap wall which accommodates the gas opening so that the opening is coaxial with the central axis, and to which the internal and external walls are connected at a location spaced from the gas opening along the central axis and along a direction perpendicular to the central axis, the internal portion being defined by the cap wall and the internal wall.

According to a second aspect of the present disclosure, there is provided a medical ventilation mask having a central axis and comprising, at least when the mask is ready for use: a mask frame having an external wall and an internal wall spaced inwardly from the external wall at least along a majority of lengths of these walls along the central axis, the internal wall defining at least partially an internal portion of the mask frame, the external wall defining at least partially an external portion of the mask frame including an auxiliary space between the walls; a gas opening connectable to a ventilation machine to provide fluid communication therewith of the internal portion of the mask frame; an internal seal connected to said internal wall at one end thereof and having an internal rim at another end thereof at which the internal seal is configured for contacting with the face of a subject so that the internal portion and the internal seal define with the face of a subject a ventilation cavity in fluid communication with the gas opening for delivering pressurized ventilation gas to the airways of the subject; an external seal connected to said external wall at one end thereof and having an external rim at another end thereof at which the external seal is configured for contacting with the face of the subject at a location spaced from the internal rim at least in a direction perpendicular to the central axis, so that said external portion with its auxiliary space and the internal seal define with the face of a subject a vacuum cavity; at least one vacuum outlet port connected to the mask frame to provide fluid communication between said auxiliary space and a vacuum line; and wherein the mask frame optionally further comprises a cap wall which accommodates the gas opening and to which the internal and external walls are connected at a location spaced from the gas opening along the central axis and along a direction perpendicular to the central axis, the internal portion being defined by the cap wall and the internal wall.

Similarly, to the mask of the first aspect, in the mask of the second aspect, the wherein the internal rim is spaced from the reference plane along the central axis to a first distance D1 and the external rim is spaced from the reference plane along the central axis to a second distance D2 longer than the first distance.

During ventilation, excess ventilation gas and exhaled breath leak from the ventilation cavity into the auxiliary space. The auxiliary space is subjected to negative pressure to thereby evacuate said excess ventilation gas and exhaled breath through the at least one vacuum outlet port into the vacuum line.

The fact that the distance D2 is greater than D1 means that the external rim protrudes farther towards the face of the subject than the internal rim, thus enabling sealing of each of the ventilation and the vacuum cavities upon fitting of the mask on the subject's face, thereby tailoring the mask to the anatomy of the human face and rendering the mask to be a dual-seal mask. In addition, the fact that the distance D2 is greater than D1 facilitates aerosols collection and scavenging during ventilating. In other words, without being bound by theory, the longer D2 distance compensates for the convexity of the human face, thus allowing better seal of both cavities and thereby environmentally safe aerosol scavenging during safe ventilation.

The existence of the two cavities in the dual-seal mask allows their operation under different pressure regimes simultaneously, i.e. it allows the operation of the ventilation cavity at a high ventilation pressure and the vacuum cavity at a negative pressure. Thereby, the mask manages aerosol clearance by suctioning at the vacuum cavity, simultaneously with, and without interrupting, the ventilation in the ventilation cavity, which thereby permits safe aerosol removal during ventilation.

As described above, the mask comprises two face-engaging rims: the internal rim and the external rim, each configured to be tightly attached onto the facial skin of the subject creating a gas-tight seal between each of the internal rim and the external rim and the facial skin, which secures against release of potentially hazardous aerosol particles to the environment while allowing uninterrupted ventilation.

Regarding the distances D1 and D2, a difference therebetween should be such as to suit natural topography of the human face where a location at which the external seal is to contact the face is further from the external portion of the mask, than a location at which the internal rim is to contact the face.

Thus, in some embodiments, the ratio D1:D2 is at least 1:1.10, more particularly it can be at least each of the following: 1:1.2, 1:1.3, 1:1.4, 1:1.5, 1:1.6, 1:1.7, 1:1.8, 1:1.9, 1:2.

By some embodiments, the ratio D1:D2 is between 1:1.1 to 1:1.3.

For example, the difference between D2 and D1 can be in the range of 5 mm-20 mm. More particularly, said difference between D2 and D1 may be 5 mm, 7 mm, 10 mm, 12 mm, 15 mm, 18 mm or 20 mm.

By some embodiments, the at least one of the internal seal and external seal is detachably attachable to the respective internal and external wall of the mask frame, optionally by a quick-connection fitting, and further optionally by snap fitting.

The mask frame can have such a shape that the internal and external walls thereof each extend from an area of the mask frame adjacent the gas opening away therefrom. Alternatively, the mask frame can comprise a cap wall which accommodates the gas opening, gas port or both, e.g. coaxially with the central axis, and to which the internal and external walls are connected at a location spaced from the gas opening along the central axis and along a direction perpendicular to the central axis, the inner portion being defined by the cap wall and the internal wall.

By some embodiments, one or each of the internal and external seals has a mounting end opposite to the sealing rims, which is configured to be securely connected to the respective internal and external wall of the mask frame. The connection can be permanent or detachable.

By other embodiments, the internal and external seals can be integrally connected to the respective internal and external walls of the mask frame, optionally by heat welding.

By other embodiments, the internal seal and external seal can be formed as unitary bodies with the mask frame.

The mask can further comprise structural reinforcement arrangement extending between the cap wall and external surface of the external wall. The arrangement can be configured for preventing the collapse of the external portion onto the internal portion of the mask frame, when vacuum is applied to the vacuum chamber.

The mask can further have engagement elements for connecting headgear or straps thereto to allow fitting and positioning the mask onto the subjects' face. Said engagement elements may be selected from headgear elements, loops configured for strap insertion and clips configured for quick connection and release, and straps. The engagement elements located on the external portion of the mask frame, can be integrally connected thereto, or unitarily formed therewith.

The structural reinforcement arrangement can be configured to ensure that pulling force applied to the mask via the engagement elements is uniformly distributed between the internal portion and the external portion, thereby facilitating the dual sealing described above. For example, the reinforcement arrangement is configured for transferring a force applied thereat upon pulling the straps or the headgear, from the external portion to the internal portion of the mask frame.

By some embodiments said arrangement comprises a plurality of reinforcement elements radially spaced from each other about the central axis. Optionally, said one or more reinforcement elements are hardened silicon ribs.

By other embodiments said reinforcement arrangement is in the form of a single continuous element.

The internal rim may comprise an internal curved lip having an internal lip extremity and an internal rim edge at which the internal lip terminates. In addition, or alternatively, the external rim may comprise a curved external lip having an external curved lip extremity and an external rim edge at which the external lip terminates. The term ‘extremity’ means an outermost point/area of the rim, i.e. its point/area spaced to a maximal extent, along the central axis, from the gas opening or reference plane defined thereby.

By some embodiments said curved internal lip is open towards the interior of the internal portion and said curved external lip is opened away from the auxiliary space. Without being bound by theory these opposite lip orientations prevent the two lips from interfering with each other, leading to compromised seal and thereby diminished mask function. Since the ventilation cavity is subjected to positive pressure, the internal lip open towards the ventilation cavity will be tightened onto the face during ventilation. The auxiliary space is subjected to negative pressure and thus the external lip opened away from the auxiliary space will be tightened into the subjects' face during ventilation.

By other embodiments said curved internal lip is open towards the interior of the internal portion and said curved external lip is open towards the auxiliary space.

Excess ventilation gas and exhaled breath may leak through spots or areas in which the internal portion frame or the external portion are weakly sealed onto the subject's face. By some embodiments said external portion has a nose bridge area.

By some embodiments, the mask comprises an additional chin area.

By some embodiments, said chin area is oppositely spaced from said nose bridge area along a direction perpendicular to the central axis.

By some embodiments, said at least one vacuum outlet port is positioned in proximity to said nose bridge area. The term proximity as defined herein encompasses a distance not exceeding 30 mm.

By some embodiments one of two vacuum outlet ports is positioned in proximity to any one or both of the nose bridge area and the chin area.

By some embodiments said mask further comprising at least one pressure regulating port providing fluid communication between said auxiliary space and an exterior of the mask.

The types and positioning of said at least one pressure regulating port may be configured for improving the gas circulation within the auxiliary space to thereby improve aerosol scavenging.

By some embodiments said at least one pressure regulating port is a basic port within the external portion, permitting an unrestricted fluid communication between the auxiliary space and the mask exterior. Without being bound by theory said basic port supports pressure regulation within the auxiliary space that is subjected to a constant negative pressure.

By some embodiments said port is located in proximity of the chin area. At times said at least one pressure regulating port is a one-way valve configured to have two states; an open state permitting gas ingress the auxiliary space and a closed state barring gas egress from the auxiliary space.

Said at least one pressure regulating port may be configured for serving two functions: (i) maintenance of a relatively constant pressure within the vacuum cavity and thus supporting gas scavenging; and (ii) ensuring that no gas will be suctioned out the ventilation cavity whereby aerosol removal is achieved without compromising the ventilation treatment effectiveness. By some embodiments, the at least one pressure-regulating valve may be any one or combination of one-way valve, a semi-closed valve, a constantly open port, or a divider valve. Said at least one valve and may be made of an elastomer such as rubber.

By some embodiments, the mask may further comprise a visual indicator for binarily or quantitatively indicating the sealing of any one or both of the ventilation cavity and the auxiliary space.

In accordance with another aspect of the present disclosure, or as an addition to any of the above aspects, there is provide a medical ventilation mask, comprising: a mask frame and a seal connected thereto and having a mask rim configured for contact with the face of a ventilated subject and defining a ventilation cavity configured for delivering pressurized ventilation gas from a gas opening to the airways of the subject and exhausting breath exhaled by the subject; and an aerosol removal arrangement comprising one or more aerosol removal ports defined in the mask frame and configured for collecting aerosol from the exhaled gas, said ports being in flow communication through one or more gas conduits with at least one vacuum outlet port for connection to a vacuum source.

Thus, gas flowing through said ports, carrying aerosol from exhaled breath, flows through the conduit system and is evacuated through the vacuum port into a vacuum line. The vacuum port or the vacuum line may comprise a trapping arrangement for trapping the aerosol particles from the evacuated gas.

The gas flowing through the gas conduit system may be made entirely of the gas evacuated from the ventilation cavity through said one or more ports. Alternatively, the gas conduit system may be configured to have a constant flow of gas therethrough, from a source other than the ventilation cavity, for example from external ports linking the conduit system with the outside and permitting air to ingress from the outside, flow through said conduit system and then out from the vacuum port. Such external ports would be typically fitted with a one-way valving arrangement to permit flow of air only into the conduit system and hinder gas egress to the exterior through such ports. Such constant flow may serve as an effective carrier of the aerosol particles and facilitate the streamlined transport of these to the vacuum port.

By some embodiments, said mask further comprises an auxiliary frame, overlaying at least a portion of the mask frame and defining said one or more gas conduits leading from the one or more aerosol removal ports to a vacuum outlet port, said auxiliary frame further comprises said at least one vacuum outlet port.

By some embodiments, said one or more gas communication conduits are defined between the auxiliary frame and the mask frame.

By some embodiments, an auxiliary space is defined between the auxiliary frame and the mask frame and comprises a vacuum port, said auxiliary space defining said one or more gas conduits, and said one or more aerosol removal ports leading from the ventilation cavity to the auxiliary space.

By some embodiments, said auxiliary frame has an auxiliary rim for contact with the face of the subject.

By some embodiments, said mask comprises a pressure-regulating valve disposed in the auxiliary frame.

By some embodiments, said vacuum port comprises a flow-control valve.

In all the above aspects, the mask frame can be rigid or semi-rigid and have a shape configured to be placed over and fully cover the airways of a human subject. By some embodiments, the mask frame is selected from nasal, oral and oral/nasal mask frame. By some embodiments, the mask has a generally concave oval or triangular shape. The mask may be made of a hard, transparent plastic such as polyethylene (PP) and polypropylene (PE). By some embodiments, the mask frame is comprised from a non-elastic material. By some embodiments, the mask is made of an elastic material. By some embodiments said elastic elastomers are selected from polyurethane, silicone, and transparent rubber. By some embodiments, the mask frame is made from any one of polycarbonate, polyethylene (PET), PP, and transparent plastic. By some embodiments, a rigid frame, or a bracing system, providing reinforcement between the cap wall and outer surface of the external wall or any one or both of the mask frame and the auxiliary frame is comprised within said mask. Said reinforcements may be obtained by material thickness, hardness, or design.

By some embodiments, the mask frame is made of a rigid material, the internal and external seals are made of an elastic material, and the connection of the internal and external walls of the mask frame to the respective internal seal and external seal is provided by a mechanical means, e.g. a quick-connection fitting, optionally by snap fitting. Alternatively, the mask frame and seals can be made of the same elastic material, wherein the mask frame is reinforced by any suitable means. For example, the thickness of the mask frame may be greater than the thickness of the seals, or the mask frame can be formed with reinforcement elements. Thus, the mask frame can be a multi-use mask or a disposable mask.

By some embodiments the mask is a single-use, disposable mask, wherein the mask frame, the internal seal and the external seal are all made of an elastic material, and wherein the internal portion and the external portion of the mask frame are integrally connected to the respective internal seal and external seal, optionally by heat welding.

In all aspects described herein, the mask may further include mask engagement elements such as headgear or straps for fitting and positioning the mask onto the subjects' face. In the mask of the first and second aspects described above, the mask engagement elements can be connected to the mask frame at a positioning region thereof located in the vicinity of an area where a distance between the internal and external walls is minimal. When the mask frame has a cap wall to which both the internal and external walls are connected, the positioning region is located at least partially on the cap wall.

In all aspects described herein, the mask is configured for NIV. The NIV mask may, for example, be configured for CPAP ventilation, and Bi-level positive airway pressure (BiPAP) ventilation.

By some embodiments, the mask is a total face mask, covering the entire face of the subject with a sealed area.

Unlike existing anesthetic masks (which handle lower gaseous pressure in compared to ventilation pressure), the mask disclosed herein is uniquely configured to support high flow and pressure (and accordingly high aerosol velocity), e.g. a flow rate of 120+ LPM while simultaneously removing aerosols.

By some embodiments, the mask is configured for use under high flow rate or ventilation pressure. By some the mask is configured for use under flow rate of at least 10 LPM, at times, at times 120+ LPM.

By some embodiments any one or both of the seals is elastic, e.g. made of silicone, thermoplastic elastomer (TPE), or polyurethane.

In all aspects described herein, term seal refers to an element configured for connection with a portion of the mask frame at one end, and for contacting with the face of a subject on the other end. In all the above aspects, the sealing described herein encompasses any face seals known in the art. By some embodiments, said pressure in any Positive End Expiratory Pressure (PEEP) known in the field of invention. At times, said pressure is selected from 3-10 cmH₂O, although it may also be higher than 10 cmH₂O, e.g. in the range of 10-65 cmH₂O.

In all the above aspects the mask described of this disclosure may be used for ventilating subjects, at times patients of different kinds and with different conditions. These include subjects suffering from a chronic medical condition or an emergency medical condition. The subjects may, for example, be such suffering from any one of obstructive sleep apnea syndrome (OSAS), acute respiratory distress syndrome (ARDS), Chronic obstructive pulmonary disease (COPD) and pulmonary edema. Other examples include subjects suffering from a microbial pathogen disease, e.g. a viral disease such as a SARS-CoV-2 (the causative virus of the COVID-19 pandemic, MERS (Middle Eastern Respiratory Syndrome) or other coronavirus infections. The subjects may also be such suffering from viral diseases and an additional pathology.

In all the above aspects, when fitted onto the subjects' face the mask frame defines a ventilation cavity, which is the space enclosed within the internal portion of the mask frame and the internal seal and the facial skin, and that is in gas communication with the subject's airways. The ventilation cavity is configured for delivering pressurized ventilation gas from a gas opening to the airways of the subject and exhausting the exhaled breath, that carries airborne aerosol particles coming from the subject's airways.

The ventilation gas may be any gas applicable in ventilating patients and may be different gas mixtures depending on the patient condition and the treatment. It may be air, oxygen-enriched air, pure oxygen, mixture of these with other gasses, etc. The ventilation gas may also comprise gaseous, aerosol or vaporized medication, such as anesthetic or analgesics. Said ventilation gas's pressure may be determined by the condition and the intended therapeutic treatment.

In all of the above aspects, the pressurized gas is delivered to said ventilation cavity from a gas port connectable to the gas opening and either directly or indirectly, to a ventilation machine. The ventilation machine may be any NIV ventilation machine, such as non-invasive positive-pressure (NIPPV) and Negative-Pressure Ventilation (NPV) machines. The ventilation machine may also be bilevel positive airway pressure (BiPAP), continuous positive airway pressure (CPAP), Automatic positive airway pressure (APAP), and adaptive servo ventilation (ASV) machines.

During the exhalation part of the ventilated breathing cycle, the ventilation cavity is filled with exhaled breath that includes both gas and aerosol (i.e. gas-borne liquid droplets), exhaled by the subject.

The aerosol particles may comprise tiny (0.01-10 μm) or larger (10-100 μm) liquid droplets, droplet nuclei that are dried-out droplets residual potentially carrying microbes. By some embodiments said aerosol comprises aerosol drug.

In all the above aspects the ventilation mask may comprise an aerosol removal arrangement comprising one or more aerosol removal ports. The term aerosol removal ports refer to openings defined in the mask frame and configured for collecting aerosol particles. Said removal ports may have variable size, shape and pattern. The diameter of said removal ports may be in the range of 0.5-10 mm.

By some embodiments, the mask comprises an array of removal ports.

Said removal ports are configured for collecting aerosol from the exhaled gas and are in flow communication through a gas conduit with at least one vacuum outlet port for connection to a vacuum source.

By some embodiments one or more of the aerosol removal ports is constituted by an undulation of the mask rim that defines a gap between the rim and the face of the subjects once the mask is in use. By some embodiments, the mask rim is configured to permit air leakage under said rim only at a defined pressure.

By some embodiments said at least one removal port is defined by at least one weakened sealing frame region. At time, the weakened region is in approximation to the subject's cheekbones, nose, or both.

By some embodiments said at least one removal ports is configured to permit flow under a certain pressure threshold or up to certain pressure threshold. This may be achieved by disposing a low-pressure safety valve, or a proportional relief valve disposed within the ports.

The term gas conduit encompasses any tube or chamber through which gas flow communication is enabled. Said communication is typically between said removal ports and said at least one vacuum outlet (either directly or via one or more merged gas conduits).

In all of the above aspects vacuum outlet port is the vent thought which aerosol is evacuated from the mask. Said vacuum port is connectable, either directly or indirectly to a vacuum source. By some embodiments said vacuum source is a medical suction machine, a pump, a central vacuum source, etc. By some embodiments, the applied negative pressure is in the range of −10 to −300 cmH₂0. The applied negative pressure may depend on the mask size, the ventilation pressure, and the type of ventilation (BiPAP or CPAP). By some embodiments, the applied negative pressure is a constant pressure. By other embodiments, the applied negative is variable negative pressure. In all of the above aspects said flow-control valve may induce vacuum in a pulsed manner. At times, said induced vacuum may be triggered by the sensing of a pre-determined pressure in any one or more of the gas ports, mask frame, ventilation cavity, auxiliary space aerosol removal port, auxiliary frame, gas conduit, and vacuum outlet port.

By some embodiments, said at least one aerosol removal ports are in proximity to the at least one vacuum port.

By some embodiments, said valve is configured to open under pre-determined pressure differential between the one or more gas conduits and the ventilation cavity.

The mask provided by this disclosure is designed to allow clearance of aerosol via suctioning while not interfering or compromising with ventilation functions. Thereby the vacuum in the aerosol removal arrangement is determined to be any value that enables concurrent pressure maintenance within the ventilation cavity. In order that the vacuum in the aerosol removal arrangement will not (or only marginally) affect the pressure in the ventilation cavity, the gas flow out of the aerosol removal ports should be controlled to be low compared to the ventilation flow. This may be achieved by controlling the size of the ports, through use of flow restrictors or by valves. By some embodiments, said medical ventilation mask further comprises at least one pressure sensor and/or flow meter in any one or more of the gas ports, mask frame, ventilation cavity, aerosol removal port, auxiliary frame, gas conduit, and vacuum outlet port.

The gas communication conduits may be defined between the auxiliary frame and the mask frame. These two frames, as already noted above, may define an auxiliary space between them. The auxiliary space may serve as the gas conduits or may be separated, by divider walls, into one or more subsidiary spaces (or sub-spaces), each constituting a gas conduits, all of which are linked to one or more vacuum port.

By some embodiments, said one or more aerosol removal ports are defined by one or more openings in the mask frame, leading from the ventilation cavity to the auxiliary frame. At times, said one or more openings are fitted with a flow-control member, e.g. in the form of pressure-regulating valve. By other embodiments said pressure-regulating valve is a one-way valve permitting unidirectional flow out of the flow only in the direction out of the ventilation cavity.

This disclosure is not limited by the number or the structural arrangements of aerosol removal ports, gas conduits, and vacuum ports and these may range in number and structure. By some embodiments, the ventilation mask comprises an array of gas conduits leading from a corresponding array of aerosol removal ports to the at least one vacuum port. At times, two or more gas conduits are in gas communication with one another And may, for example, merge with one another into a combined conduit linking these gas conduits to the vacuum port.

By some embodiments the mask comprising an auxiliary space defined between the auxiliary frame and the mask frame divided into an array of sub-spaces, each of the sub-spaces having at least associated aerosol removal port between it and the ventilation cavity, and each of the subspaces being in flow communication with the at least one vacuum outlet port.

In all of the above aspects the mask is configured for use in non-invasive ventilation (NIV). At times, said mask is configured for bilevel positive airway pressure (BiPAP), continuous positive airway pressure (CPAP), Automatic positive airway pressure (APAP), and adaptive servo ventilation (ASV) machine.

In all aspects described herein, the mask the mask may comprise a UV purifier.

In all aspects described herein, the mask the mask may comprise a filter, within the at least one vacuum outlet port. At times said filter is a viral filter for capturing viruses. At time said filter is a SARS-CoV-2 filter.

By some embodiments, the masks further comprise an alarm element, for alarming compromised sealing in any portion of the mask (e.g. ventilation cavity, auxiliary space) when fitted onto the subjects' face.

### BRIEF DESCRIPTION OF THE DRAWINGS

In order to better understand the subject matter that is disclosed herein and to exemplify how it may be carried out in practice, embodiments of a ventilation mask will now be described, by way of non-limiting examples only, with reference to the accompanying drawings, in which, unless indicated specifically, the ventilation mask is shown in its state ready for use:

FIGS. 1A, 1B, 1C and 1D are, respectively, schematic front, perspective, longitudinal cross-sectional, and side view of a ventilation mask according to an exemplary embodiment of the presently disclosed subject matter.

FIGS. 2A, 2B, 2C and 2D are, respectively, schematic front, rear, upper and exploded view of a mask according to another exemplary embodiment of the presently disclosed subject matter.

FIGS. 3A, 3B, 3C, 3D and 3E are, respectively, schematic side, top, perspective, and two longitudinal cross-sectional view of a mask according to another exemplary embodiment of the presently disclosed subject matter.

FIG. 4 is a schematic side view of the mask shown in FIGS. 3A to 3E, when fitted onto a subject's face.

FIG. 5 is schematic side view of a ventilation mask according to a further exemplary embodiment of the presently disclosed subject matter.

FIG. 6 is schematic side view of a ventilation mask fitted onto the subject' face, according to still further exemplary embodiment of the presently disclosed subject matter.

FIG. 7 is schematic rear view of a mask, which can be any of the masks shown in the previous drawings.

FIG. 8 is a schematic enlarged side view of a portion of a mask, which any of the masks shown in the previous drawings can have, according to an exemplary embodiment of the presently disclosed subject matter.

FIGS. 9A-9C are, respectively, schematic upper, rear and perspective view of a ventilation mask according to a still further exemplary embodiment of the presently disclosed subject matter.

### DETAILED DESCRIPTION OF EMBODIMENTS

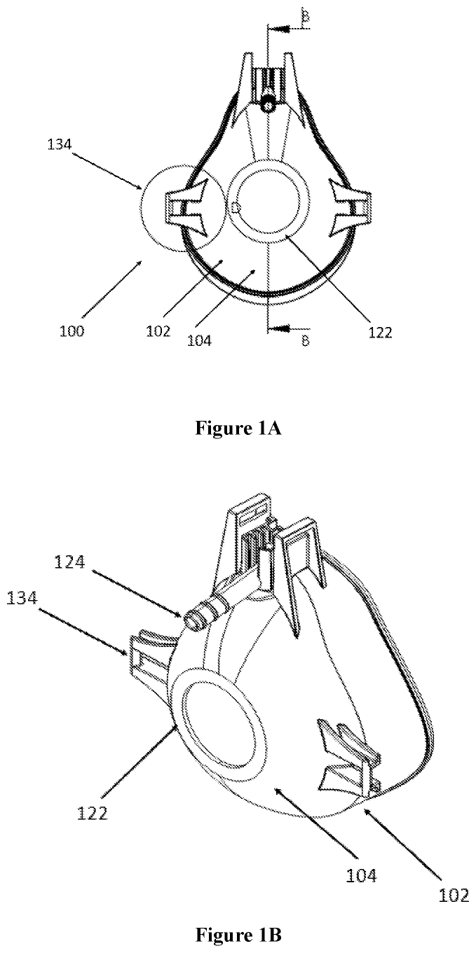

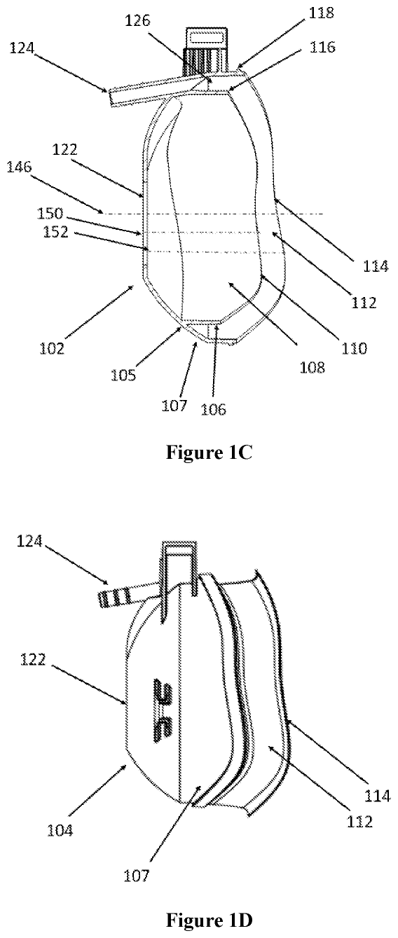

A schematic illustration of an aerosol-proof ventilation mask 100 of an exemplary embodiment of this disclosure is shown in FIGS. 1A-1D, wherein FIGS. 1A-1D show the mask in isolation in respective front, perspective longitudinal cross-section and side view. The mask 100 allows safe ventilation by collecting and removing aerosols exhaled by the subject during ventilation. When fitting the mask 100 onto the subject's face during ventilation, viral aerosols are scavenged to a vacuum port 124 that is connectable to a vacuum source (not shown). As best seen in FIG. 1C, the mask 100 has a mask frame 102 with cap wall 104 defining the front of the mask and having an opening 122 coaxial with a central axis 146 of the mask and defining a reference plane of the mask, at which a gas opening 122 is mounted, an internal wall 116 defining with the cap wall an internal portion 106 of the mask frame, an external wall 118 overlaying the internal wall 116 so as to leave an auxiliary space 126 therebetween, the external wall and the auxiliary space defining an external portion 107 of the mask frame. The internal and external walls merge with the cap wall at an area 105 spaced from the gas opening both along the central axis and in a direction perpendicular to the central axis.

The vacuum outlet port 124 is optionally positioned in proximity of the nose bridge area and may comprise a filter (not shown).

The mask 100 further comprises an external seal 112 connected to the external wall 118 at one end thereof and having an external rim 114 at the other end thereof, configured for contacting with the face of the subject so that the external portion of the mask frame with its auxiliary space 126 defines therewith a vacuum cavity (designated as 442 in FIG. 4).

The mask 100 further comprises an internal seal 108 connected to the internal wall 116 at one end thereof and having an internal rim 110 at the other end thereof configured for contacting with the face of a subject so as to define therewith a ventilation cavity (designated as 440 in FIG. 4) in fluid communication with the gas opening 122 for delivering pressurized ventilation gas to the airways of the subject. The gas opening 122 is at the front end of the cap wall 104 of the mask frame 102.

In other words, the mask 100 comprises two face-engaging rims: the internal rim 110 and the external rim 114, each configured to be tightly attached onto the facial skin of the subject creating together a gas-tight seal between the mask and the facial skin, which secures against release of potentially hazardous aerosol particles to the environment while allowing uninterrupted ventilation.

With reference to FIG. 4, during ventilation at the ventilation cavity, excess ventilation gas and exhaled breath leak from the ventilation cavity 440 into the vacuum cavity 442 via gaps created between the internal rim of the internal seal and the face of a subject due to the impossibility of ideal sealing therebetween (not shown).

The gas opening 122 is connectable to a ventilation machine and the vacuum port 124 is connectable to a vacuum line (not shown) Thus, in operation the ventilation cavity is subjected to a positive pressure for delivering pressurized ventilation gas to the airways of the subject, and the vacuum cavity is subjected to negative pressure to thereby evacuate excess ventilation gas and exhaled breath therefrom through the vacuum port 124.

Reverting to FIG. 1C, the internal rim 110 and the external rim 114 are spaced from the gas opening 122 defining the reference plane, along the central axis, to respective distances D1 150 and D2 152, the latter distance being longer than the former distance. The difference between D1 150 and D2 152 can suit the human face topography where a location at which the external rim 114 is to contact the face is further from the gas opening 122 or the reference plane, than a location at which the internal rim 110 is to contact the face. Since external rim 114 protrudes farther towards the face of the subject than the internal rim 110, potentially hazardous aerosol particles escaping the ventilation cavity with excess ventilation gas and exhaled breath are prevented by the external wall of the mask frame from reaching the external rim of the mask frame before they are removed from the vacuum cavity. Thereby, the mask 100 is a dual-seal mask having a sealed ventilation cavity and a sealed vacuum cavity upon fitting of the mask on the subject's face. The resulted dual-seal configuration allows environmentally safe aerosol scavenging during safe ventilation.

The internal wall 116 and the external wall 118 are formed as a unitary body with the cap wall 104. The internal seal 108 and external seal 112, can also be formed as a unitary body with the internal and external walls, but they can alternatively be connected to these walls integrally, e.g. by heat welding or by any mechanical means.

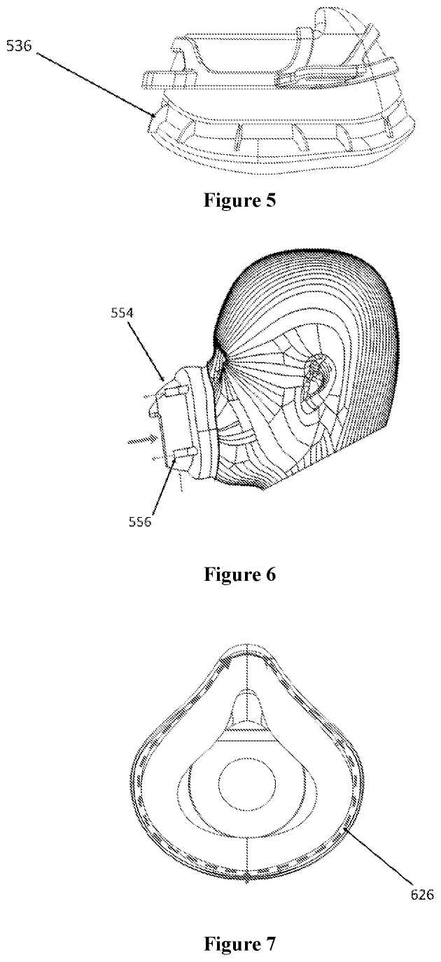

The mask 100 may further comprise structural reinforcement arrangement extending between the cap wall 104 and outer surface of the external wall 118. One example of such arrangement is shown in FIG. 5, where it is designated as 536, where it shown to comprise a plurality of reinforcement elements spaced from each other.

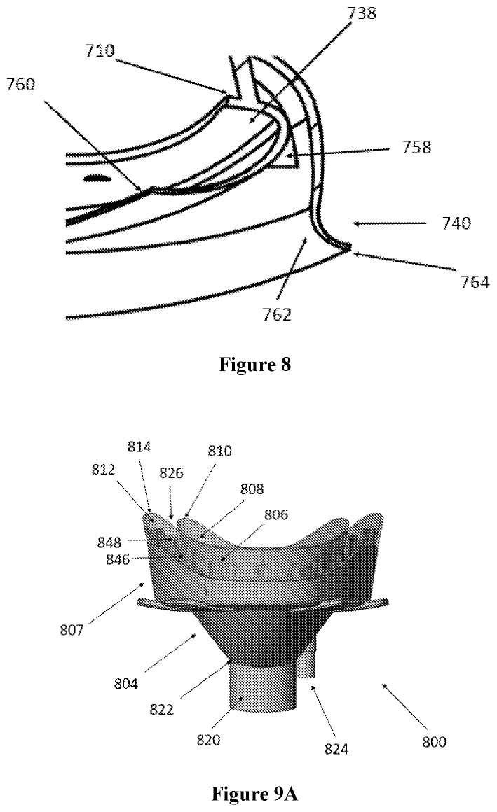

As illustrated in FIG. 8, the internal rim 710 which can comprise a curved internal lip 738 and the external rim 714 which can comprise a curved external lip illustrated as 740. In this example, the inner curved lip 738 is open inwardly, i.e. into the interior of the ventilation cavity, and the external curved lip 740 is open outwardly, i.e. towards the exterior of the mask when the mask is in use. More detailed description of the internal and external lips is presented below in the detailed description of FIG. 8.

The external portion 107 of the mask frame can have a nose bridge area, and optionally a chin area oppositely spaced from the nose bridge area. The mask 100 may further comprise a pressure regulating port (not shown) providing fluid communication between the external portion 107 and an exterior of the mask, located in proximity of the chin area.

Mask 100 also includes mask engagement elements 134 that may be linked to straps for fixing the mask to the subject's face over the airways. The mask 100 may be configured for use in non-invasive ventilation (NIV), (CPAP) ventilation, or (BiPAP) ventilation, and may further comprise a UV purifier.

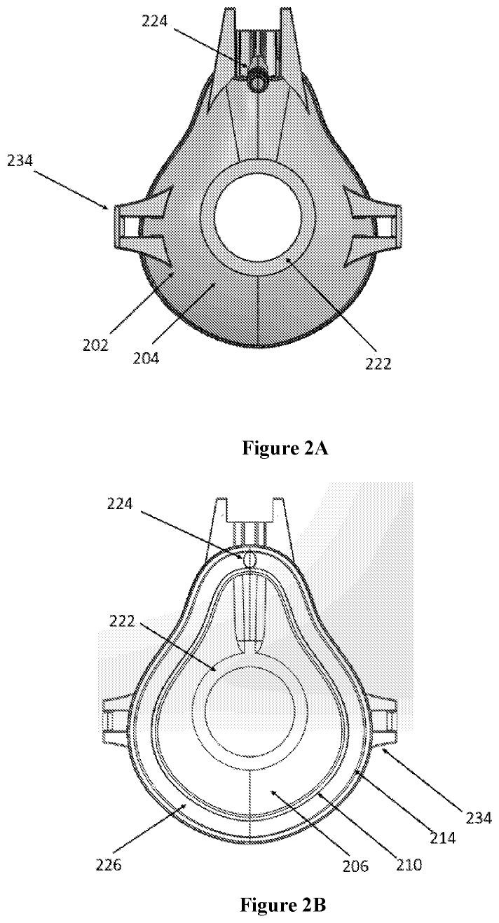

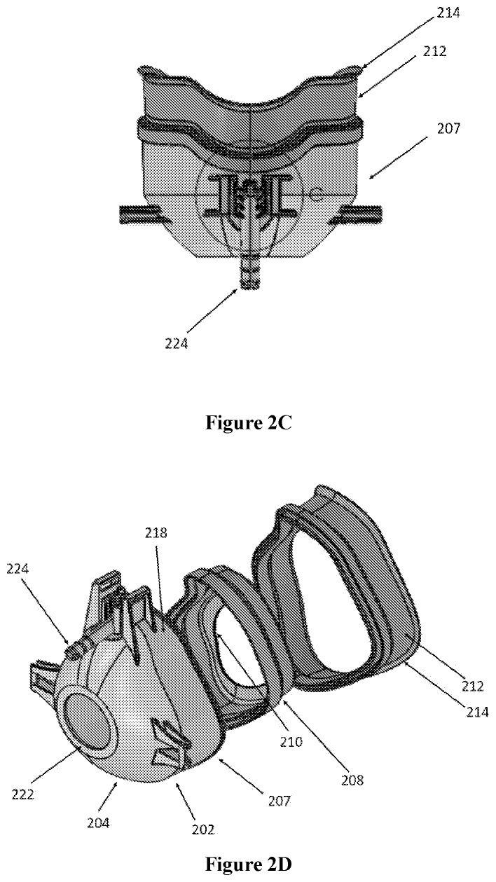

An exemplary embodiment of a mask 200 is shown schematically in FIGS. 2A-2B, which has the same elements as in the mask 100 described above.

In these figures like reference numerals shifted by 100 are used for elements of the mask 200 which are seen there and which function in the same way as those of the mask 100 described above with reference to FIGS. 1A-1D. The reader is referred to the description of these elements in the mask 100. For example, in FIGS. 2A-2C a mask frame 202 is shown with its internal portion 206 and external portion 207 comprising an auxiliary space 226, which are the same as the mask frame 102 with its internal portion 106 and external portion 107 with its auxiliary space 126.

As shown in FIG. 2C, the mask 200 further comprises an internal seal 208 having an internal rim 210 for contacting with the face of a subject so as to define therewith a ventilation cavity in fluid communication with the gas opening 222 for delivering pressurized ventilation gas to the airways of the subject, and external seal 212 connected to an external wall 218 of the mask frame and having an external rim 214 configured for contacting with the face of the subject for creating a vacuum cavity 232 in fluid communication with a vacuum port 224 for removing aerosols exhaled by the subject during ventilation and escaped with excess ventilation gas and exhaled breath leak from the ventilation cavity into the vacuum cavity.

FIG. 2D illustrates an exploded view of the mask 200, comprising the mask frame 202 with its cap wall 204, external wall 218, internal seal 208 to be connected to an internal wall of the mask frame (not seen in FIG. 2D), and external seal 212 configured to be connected to the external wall 216 by any suitable mechanical connection means, as explained above with reference to the mask 100 or as described below with respect to a mask 300.

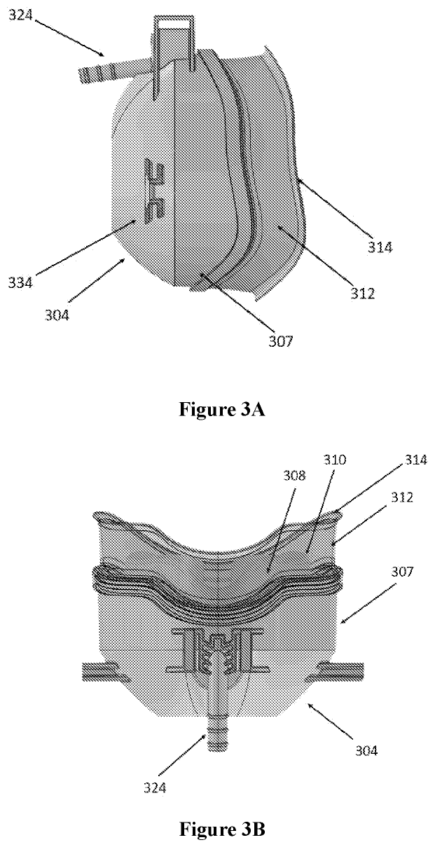

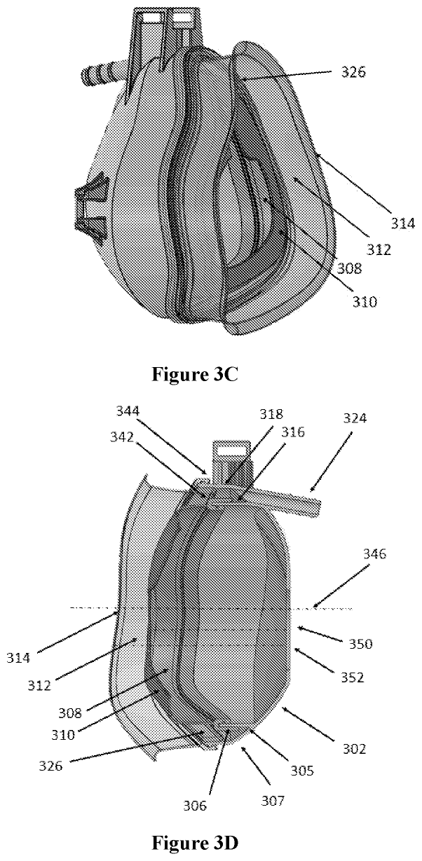

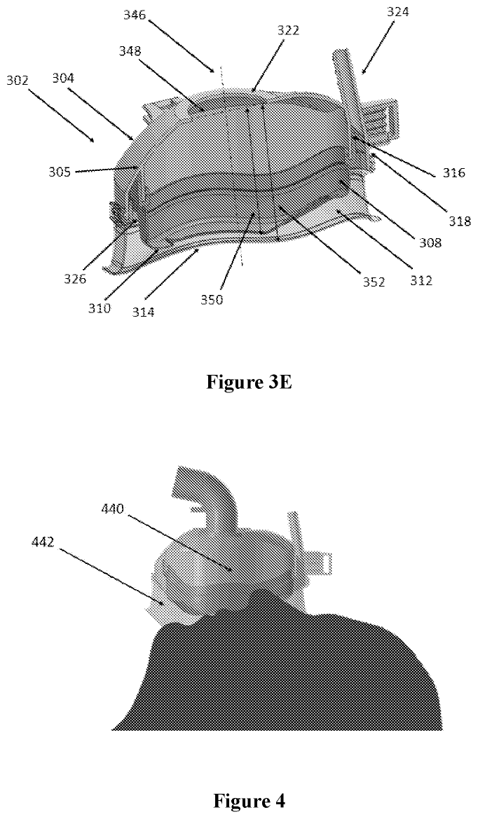

A mask 300 is shown schematically in FIGS. 3A-3E and it has the same elements as the masks 100 and 200 described above. In these figures like reference numerals shifted by 200 are used for elements of the mask 300, which are seen in these figures and which function in the same way as those of the mask 100 described above with reference to FIGS. 1A-1D. The reader is referred to the above description of these elements in the mask 100.

FIG. 3E presents a longitudinal cross-sectional view of the mask 300 having a central axis 346 and comprising a mask frame 302 with a cap wall 304, external wall 318, internal wall 316, internal seal 308 and external seal 312. The cap wall is formed with an opening 322 configured for connecting a gas port thereto (not shown) and with a vacuum port 324 open into an auxiliary space 326.

The opening 322 lies in or defines a reference plane RP 348 perpendicular to the central axis 346. An internal rim 310 of the internal seal 312 is spaced from the reference plane RP along the central axis 346 to a first distance D1 350 and the external rim 314 of the external seal 308 is spaced from the reference plane RP along the central axis 346 to a second distance D2 352 longer than the first distance D1 350.

The internal and external walls merge with the cap wall at an area 305 spaced from the reference plane both along the central axis 346 and in a direction perpendicular to this axis. Each of the internal and external walls have a mounting end 346, 348 respectively, to which the internal and external seals are mountable.

Each of the internal and external seals has a mounting end designated as 342, and 344 respectively, opposite to the sealing rims of the seal, which is securely connected to the the corresponding internal and external wall of the mask frame. The connection can be permanent or detachable.

Reverting to FIG. 4, it illustrates the dual-seal function of each one of the masks 100, 200 and 300 described above, when the mask is fitted onto the subject's face with the internal and external rims of its seals in tight contact with the face of the subject, and with the gas port connected to a ventilation machine and the vacuum port being connected to a vacuum line (both not shown). The dual-seal function is provided by the following two cavities created by the mask with the subject's face once the mask is fitted thereto as described above: the ventilation cavity 440 in fluid communication with the gas opening for delivering pressurized ventilation gas to the airways of the subject, and the vacuum cavity 442 in fluid communication with the vacuum port for removing aerosols exhaled by the subject during the ventilation and escaped with excess ventilation gas and exhaled breath leak from the ventilation cavity into the vacuum cavity.

FIG. 5 illustrates a ventilation mask which can be any of the masks described above, having a structural reinforcement arrangement in the form of an array of silicon rims 536 on an outer surface of the external wall of the mask frame. Such reinforcement may be implemented in a single use mask that is comprised of an elastic material to support the dual-seal of the mask onto the subjects' face. The illustrated reinforcement arrangement enables safe and controlled tightening of the internal portion of the mask when pulling the straps connected to the engagement elements, thereby allowing a safe and secured dual-seal.

Any of the above-described masks can have two vacuum ports, as illustrated in FIG. 6, in which the right-pointing arrow illustrates incoming ventilation gas and the two left-pointing arrows represent suctioned aerosol and exhaled gas from two vacuum ports 554 and 556, the two vacuum ports are positioned in proximity to weak sealing spots beings nose bridge and chin areas of the mask.

FIG. 7 depicts a rear view of a mask which can be any of the masks 100, 200 and 300 described above, with arrows schematically representing exhaled breath flow pattern within the auxiliary space designated as 626 to the vacuum outlet port.

Reverting to FIG. 8, it depicts an enlarged view of a portion of any of the masks 100, 200 and 300, according to a non-limiting embodiment, which portion comprises a part of the internal and the external rim with the respective internal curved lip 738 and external curved lip 740. As seen in FIG. 8, each curved lip has an extremity and a rim edge at which the lip terminates, wherein internal curved lip 738 is open towards the interior of the mask or towards the ventilation cavity, when the mask is in use, and the external curved lip 740 is open towards the exterior of the mask or away from the ventilation and vacuum cavities, when the mask is in use. This means that the rim edge 760 of the internal curved lip is disposed within the interior of the mask or the ventilation cavity when the mask is in use, and the rim edge 764 of the external curved lip is disposed out of the any interior space of the mask and of the vacuum cavity when the mask is in use. The extremities 758 and 762 of the respective lips 738 and 740 are spaced from the reference plane of the mask (not shown in this figure) to respective distances D1 and D2 described above in the description of the masks 100 and 300.

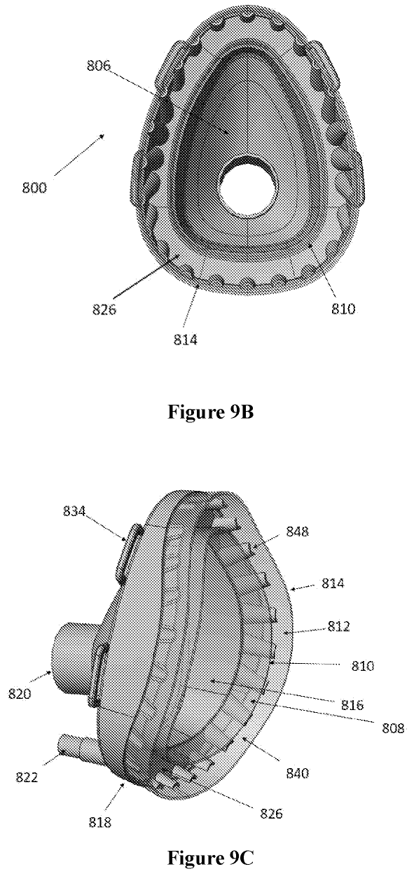

A yet another exemplary embodiment of a mask 800 is shown schematically in FIGS. 9A-9C representing the upper, rear and perspective views of the mask, respectively. In these figures like reference numerals shifted by 700 are used for elements having a similar function to those of FIGS. 1A-1D. The reader is referred to the description of FIGS. 1A-1D, where needed, for explanation of their function. For example, reference numerals 108 and 808 are used to refer to the internal seals of the respective mask frames 100 and 800, and reference numerals 112 and 812 are used to refer to the external seals of these masks. In this embodiment, which is applicable to any of the masks described above, the mask 800 comprises a plurality of gas conduits 846 formed at the periphery of the auxiliary space 826 between the internal wall 816 and the external wall 818 of the mask frame 802 configured to function as described in the General Description part of the present specification.

1-16. (canceled)

17. A medical ventilation mask having a central axis, the medical ventilation mask comprising, at least when the mask is ready for use:
a gas opening configured to be connected, in fluid communication to a ventilation machine and defining a location of a reference plane perpendicular to the central axis;
a mask frame having an internal portion and an external portion and accommodating the gas opening so as to provide fluid communication of the gas opening with the internal portion, the external portion overlaying said internal portion at least along a part of an extension thereof along the central axis and including an auxiliary space therebetween;
an internal seal connected to said internal portion at one end of the seal and having an internal rim at another end thereof, and configured for contacting with the face of a subject so that the internal portion of the mask frame and the internal seal define with the face of a subject a ventilation cavity in fluid communication with the gas opening for delivering pressurized ventilation gas to the airways of the subject, said internal rim being spaced from the reference plane along the central axis to a first distance D1;
an external seal connected to said external portion at one end of the seal and having an external rim at another end thereof, and configured for contacting with the face of the subject at a location spaced from the internal rim in a direction perpendicular to the central axis, so that said external portion of the mask frame with its auxiliary space and the external seal define with the face of a subject a vacuum cavity, said external rim being spaced from the reference plane along the central axis to a second distance D2 longer than the first distance; and
at least one vacuum outlet port connected to the mask frame so as to provide fluid communication between said auxiliary space and a vacuum line, so as to provide aerosol clearance by applying vacuum to the vacuum cavity, simultaneously with, and without interrupting, the ventilation in the ventilation cavity.

18. The medical ventilation mask of claim 17, wherein the mask frame has an external wall and an internal wall spaced inwardly from the external wall in a direction perpendicular to the central axis, the external wall defining at least partially said external portion of the mask frame, and the internal wall defining at least partially said internal portion of the mask frame, and wherein the mask frame optionally further comprises a cap wall which accommodates the gas opening so that the opening is coaxial with the central axis, and to which the internal and external walls are connected at a location spaced from the gas opening along the central axis and along a direction perpendicular to the central axis, the internal portion being defined by the cap wall and the internal wall.

19. The medical ventilation mask of claim 17, wherein a ratio D1:D2 is at least 1:1.10.

20. The medical ventilation mask of claim 17, wherein said internal rim comprises a curved internal lip open towards an interior of the internal portion.

21. The medical ventilation mask of claim 17, wherein the external rim comprises a curved external lip.

22. The medical ventilation mask of claim 21, wherein said curved external lip is open away from the auxiliary space.

23. The medical ventilation mask of claim 21, wherein said curved external lip is open towards the auxiliary space.

24. The medical ventilation mask of claim 17, wherein the external portion has a nose bridge area.

25. The medical ventilation mask of claim 24, wherein the external portion comprises a chin area oppositely spaced from the nose bridge area along a direction perpendicular to the central axis.

26. The medical ventilation mask of claim 24, wherein said vacuum outlet port is positioned in proximity of the nose bridge area.

27. The medical ventilation mask of claim 25, further comprising a pressure regulating port located in proximity of the chin area and providing fluid communication between said external portion and an exterior of the mask.

28. A medical ventilation mask having a central axis, the medical ventilation mask comprising, at least when the mask is ready for use:
a mask frame having an external wall and an internal wall spaced inwardly from the external wall at least along a majority of lengths of these walls along the central axis, the internal wall defining at least partially an internal portion of the mask frame, the external wall defining at least partially an external portion of the mask frame including an auxiliary space between the walls;
a gas opening connectable to a ventilation machine to provide fluid communication therewith of the internal portion of the mask frame;
an internal seal connected to said internal wall at one end thereof and having an internal rim at another end thereof at which the internal seal is configured for contacting with the face of a subject so that the internal portion and the internal seal define with the face of a subject a ventilation cavity in fluid communication with the gas opening for delivering pressurized ventilation gas to the airways of the subject;
an external seal connected to said external wall at one end thereof and having an external rim at another end thereof at which the external seal is configured for contacting with the face of the subject at a location spaced from the internal rim at least in a direction perpendicular to the central axis, so that said external portion with its auxiliary space and the internal seal define with the face of a subject a vacuum cavity;
at least one vacuum outlet port connected to the mask frame to provide fluid communication between said auxiliary space and a vacuum line so as to provide aerosol clearance by applying vacuum to the vacuum cavity, simultaneously with, and without interrupting, the ventilation in the ventilation cavity; and
wherein the mask frame optionally further comprises a cap wall which accommodates the gas opening and to which the internal and external walls are connected at a location spaced from the gas opening along the central axis and along a direction perpendicular to the central axis, the internal portion being defined by the cap wall and the internal wall.

29. The medical ventilation mask of claim 28, wherein the internal rim is spaced from the reference plane along the central axis to a first distance D1 and the external rim is spaced from the reference plane along the central axis to a second distance D2 longer than the first distance.

30. The medical ventilation mask of claim 28, wherein at least one of the internal seal and external seal is detachably attachable to the respective internal and external wall of the mask frame.

31. The medical ventilation mask of claim 28, further comprising a structural reinforcement arrangement extending between the cap wall and outer surface of the external wall.

32. The medical ventilation mask of claim 31, wherein said structural reinforcement arrangement comprises a plurality of reinforcement elements radially spaced from each other about the central axis.

33. The medical ventilation mask of claim 30, wherein at least one of the internal seal or the external seal is detachably attachable to the respective internal and external wall of the mask frame by a quick-connection fitting and further optionally by snap fitting.
